(19) United States
(12) Patent Application Publication
(10) Pub. No.: US 2005/0197653 A1
(43) Pub. Date: Sep. 8, 2005

(54) Filter assembly with microfabricated filter element

(76) Inventors: Jeffry J. Sniegowski, Tijeras, NM (US); Paul J. McWhorter, Albuquerque, NM (US)

Correspondence Address: MARSH, FISCHMANN & BREYFOGLE LLP, 3151 SOUTH VAUGHN WAY, SUITE 411, AURORA, CO, 80014, US

(21) Appl. No.: 10/858,153

(22) Filed: Jun. 1, 2004

Related U.S. Application Data
(63) Continuation-in-part of application No. 10/791,396, filed on Mar. 2, 2004.

(51) Int. Cl. A61K 9/22

(52) U.S. Cl. 604/891.1

(57) ABSTRACT

An implant (98) having an implant filter assembly (92) is disclosed. This implant filter housing (92) is disposed within a passageway (102) of an implant housing (100). One configuration for the implant filter assembly (92) is in the form of a filter assembly (26). The filter assembly (26) includes a LIGA filter element (22) that is disposed between adjacent ends of a first inner housing (34) and a second inner housing (38). These housings (34, 38) are at least partially disposed within an outer housing (30) such that the outer housing (30) is disposed about the LIGA filter element (22). Preferably, at least the outer housing (30) is more rigid than the implant housing (100), at least at a time when the implant (98) is installed in the target biological material.

### CROSS REFERENCE TO RELATED APPLICATIONS

This patent application is a continuation-in-part of, and claims priority under 35 U.S.C. §120 to, U.S. patent application Ser. No. 10/791,396, that is entitled “MEMS FLOW MODULE WITH FILTRATION AND PRESSURE REGULATION CAPABILITIES,” that was filed on Mar. 2, 2004, and the entire disclosure of which is incorporated by reference in its entirety herein.

### FIELD OF THE INVENTION

The present invention generally relates to the field of microfabricated devices and, more particularly, to a filter assembly that uses a filter element that is microfabricated, preferably using at least part of a LIGA process.

### BACKGROUND OF THE INVENTION

High internal pressure within the eye can damage the optic nerve and lead to blindness. There are two primary chambers in the eye—an anterior chamber and a posterior chamber that are generally separated by a lens. Aqueous humor exists within the anterior chamber, while vitreous humor exists in the posterior chamber. Generally, an increase in the internal pressure within the eye is caused by more fluid being generated within the eye than is being discharged by the eye. The general consensus is that it is the fluid within the anterior chamber of the eye that is the main contributor to an elevated intraocular pressure.

One proposed solution to addressing high internal pressure within the eye is to install an implant. Implants are typically directed through a wall of the patient's eye so as to fluidly connect the anterior chamber with an exterior location on the eye. There are a number of issues with implants of this type. One is the ability of the implant to respond to changes in the internal pressure within the eye in a manner that reduces the potential for damaging the optic nerve. Another is the ability of the implant to reduce the potential for bacteria and the like passing through the implant and into the interior of the patient's eye.

### BRIEF SUMMARY OF THE INVENTION

The present invention is embodied by a filter assembly having at least one housing and a filter element that is microfabricated, preferably using at least part of a LIGA process. More specifically, the filter element may be fabricated at least in part using short wavelength light in relation to the definition of pores through a photosensitive material (e.g., using at least part of a LIGA process). This filter assembly may be used in any appropriate application. However, one particularly desirable application for the filter assembly is implants. The present invention will be described with regard to this particular application. However, it should be appreciated that the filter assembly to be described herein may be presented independent of any application requirement(s).

A first aspect of the present invention is generally directed to a method for making an implant. The method includes fabricating a filter element. This fabrication entails using at least part of a LIGA process to form a plurality of pores that extend completely through the filter element. This filter element is disposed in a passageway of an implant housing. A flow or other migration through the implant passageway is directed through the filter element.

Various refinements exist of the features noted in relation to the first aspect of the present invention. Further features may also be incorporated in the first aspect of the present invention as well. These refinements and additional features may exist individually or in any combination. The subject first aspect may include assembling the filter element and a first housing into a filter assembly in a manner such that the filter element is maintained in a fixed position relative to the first housing. This filter assembly may then be disposed into the passageway of the implant housing. Preferably, this first housing protects and/or supports the filter element. The filter element may have first and second primary surfaces that are separated by a distance of no more than about 50 μm in one embodiment, by a distance of no more than 15 μm in another embodiment, and by a distance of about 5 μm in yet another embodiment (e.g., the filter element may be disk-shaped). The plurality of pores may extend between these first and second primary surfaces.

The above-noted first housing may be more rigid than the implant housing, at least when the implant is installed in the targeted biological material. The filter element may be disposed within the first housing such that the first housing is disposed about (e.g., “surrounds”) the filter element. For instance, the filter element and first housing may be concentrically disposed, with the filter element being disposed radially inwardly of the first housing. The filter element also may be mounted in any appropriate manner (e.g., chemically bonded) on a first open end of the first housing.

The above-noted filter element may be fabricated using at least part of a LIGA process. In this case, a photosensitive material could be patterned and developed to define a filter element mold from the photosensitive material, and an appropriate material (e.g., a metal) could be deposited (e.g., electroplated) within this mold to define a positive image of the filter element. The filter element mold would then be removed (e.g., dissolved). Another option for using at least part of a LIGA process would be to fabricate the filter element by exposing a photosensitive material to light having a short wavelength, including without limitation x-rays, extreme ultraviolet rays, or deep ultraviolet rays. The photosensitive material could then be developed (e.g., exposed portions of the photosensitive material being dissolved in the case of a positive resist system, and un-exposed portions of the photosensitive material being dissolved in the case of a negative resist system) such that the body of the filter element would be defined by the relevant portion of the photosensitive material that remains after the development. Short wavelength light is preferred in either case due to the enhanced dimensional control that may be realized.

The subject first aspect may include assembling the filter element, a first housing, and a second housing into a filter assembly. The filter element may be disposed within the second housing (e.g., such that the second housing is disposed about the perimeter of the filter element). At least part of the second housing may be disposed within the first housing. In one embodiment, the filter element is disposed within the second housing before the second housing is disposed within the first housing. In any case, this filter assembly may then be disposed into the passageway of the implant housing. Preferably, the first and/or second housing protects and/or supports the filter element. In one embodiment, the filter element in this case again may have first and second primary surfaces that are separated by the above-noted spacing limitations and between which the pores extend.

The above-noted second housing may be maintained in a fixed position relative to the first housing in any appropriate manner (e.g., press fit, shrink fit, bonded). The filter element may be maintained in a fixed position relative to the second housing in any appropriate manner as well. Preferably, the filter element and second housing are maintained in a fixed position relative to each other, as are the first and second housings. The first and/or second housings may be fabricated using at least part of a LIGA process (e.g., to define a mold from a photosensitive material in which a material is thereafter deposited to define the body of the relevant housing), or by exposing an appropriate photosensitive material to light having a short wavelength (e.g., to define the relevant housing directly from the photosensitive material), or both. Moreover, one or both of the first and second housings may be more rigid than the implant housing, at least when the implant is installed in the targeted biological material.

The subject first aspect may include assembling the filter element, a first housing, a second housing, and a third housing into a filter assembly. A first end of the second housing and a first end of the third housing each may be disposed within the first housing, such that the first housing will be disposed about the filter element when the same is disposed between the first end of the second and third housings. Any order may be used for disposing the first end of the second and third housings into the first housing. In one embodiment, the filter element is positioned on the first end of the second housing before being disposed within the first housing. In any case, this filter assembly may then be disposed in the passageway of the implant housing. Preferably, the first, second, and third housings collectively protect and/or support the filter element. In one embodiment, the filter element in this case again may have first and second primary surfaces that are separated by the above-noted spacing limitations and between which the pores extend.

The above-noted second and third housings each may be maintained in a fixed position relative to the first housing in any appropriate manner (e.g., press fit, shrink fit, bonded). The filter element may be maintained in a fixed position relative to both the second and third housings in any appropriate manner as well. For instance, the filter element may be maintained in a fixed position merely by interfacing with both the second and third housings when positioned within the first housing. Another option would be to mount the filter element to the first end of one or both of the second and third housings before disposing the same within the first housing. Preferably, the filter element is maintained in a fixed position relative to each of the first, second, and third housings.

The first, second, and/or third housings may be fabricated using at least part of a LIGA process (e.g., to define a mold from a photosensitive material in which material is thereafter deposited to define the body of the relevant housing), by exposing an appropriate photosensitive material to light having a short wavelength (e.g., to define the relevant housing directly from the photosensitive material), or both. Moreover, one or more of the first, second, and third housings may be more rigid than the implant housing, at least when the implant is installed in the targeted biological material.

A second aspect of the present invention is generally directed to a method for making an implant. The method includes fabricating both a filter element and a first housing for a filter assembly by exposing a photosensitive material to light having a short wavelength, including without limitation x-rays, extreme ultraviolet rays, or deep ultraviolet rays. After the filter element and first housing are each fabricated using this type of exposure, they are assembled into a filter assembly. This filter assembly is then disposed in a passageway of an implant housing. A flow or other migration through this passageway is directed through the filter element.

Various refinements exist of the features noted in relation to the second aspect of the present invention. Further features may also be incorporated in the second aspect of the present invention as well. These refinements and additional features may exist individually or in any combination. Preferably, the first housing protects and/or supports the filter element. In one embodiment, the filter element has first and second primary surfaces that are separated by the spacing limitations noted in relation to the first aspect and between which a plurality of pores extend.

The first housing associated with the second aspect may be more rigid than the implant housing, at least when the implant is installed in the targeted biological material. The filter element may be disposed within the first housing such that the first housing is disposed about (e.g., “surrounds”) the filter element. For instance, the filter element and first housing may be concentrically disposed, with the filter element being disposed radially inwardly of the first housing. The filter element also may be mounted in any appropriate manner (e.g., chemically bonded) on a first open end of the first housing. In any case, the first housing may include a first passageway, and the filter element may be disposed at least somewhere within this first passageway.

The subject second aspect may include assembling the filter element, the first housing, and a second housing into a filter assembly. In one embodiment, the filter element is maintained in a fixed position relative to the second housing, at least part of the second housing is disposed within the first housing such that the first housing is disposed about the filter element, and the second housing is maintained in a fixed position relative to the first housing. In another embodiment, the filter element is disposed within the second housing, and at least part of the second housing is disposed within the first housing. In either case, such a filter assembly may have the following characteristics, individually or in any combination: 1) the position of the filter element relative to the second housing may be fixed before disposing the second housing at least partially within the first housing, and the filter assembly thereafter may then be disposed into the passageway of the implant housing; 2) the filter element may be maintained in a fixed position relative to the second housing in any appropriate manner; 3) the second housing may be maintained in a fixed position relative to the first housing in any appropriate manner (e.g., press fit, shrink fit, bonded); 4) the filter element may have first and second primary surfaces that are separated by the spacing limitations noted in relation to the first aspect and between which a plurality of pores extend; 5) one or both of the first and second housings may be more rigid than the implant housing, at least when the implant is installed in the targeted biological material; and 6) the first and/or second housings may be fabricated using at least part of a LIGA process, by exposing an appropriate photosensitive material to light having a short wavelength, or both.

The subject second aspect further may include assembling the filter element, the first housing, a second housing, and a third housing into a filter assembly. Such a filter assembly may have the following characteristics, individually or in any combination: 1) the second and third housings each may be maintained in a fixed position relative to the first housing in any appropriate manner (e.g., press fit, shrink fit, bonded); 2) the position of the filter element may be fixed relative to each of the first, second, and third housings in any appropriate manner (e.g., merely by interfacing with both the second and third housings when positioned within the first housing; by mounting the filter element on an open end of one or both of the second and third housings before disposing the same within the first housing); 3) the filter element may be positioned on an open end of the second housing before being directed into the first housing; 4) the filter element may have first and second primary surfaces that are separated by the spacing limitations noted in relation to the first aspect and between which a plurality of pores extend; 5) one or more of the first, second, and third housings may be more rigid than the implant housing, at least when the implant is installed in the targeted biological material; and 6) one or more of the first, second, and third housings may be fabricated using at least part of a LIGA process, by exposing an appropriate photosensitive material to light having a short wavelength, or both.

Both the filter element and at least one housing of the filter assembly are fabricated using an exposure of a photosensitive material to light having a short wavelength in the case of the second aspect. In one embodiment, short wavelength light is used to form a mold in a photosensitive material such that the material that is subsequently deposited within this mold defines the body of the relevant filter element or filter assembly housing (the “LIG” of a LIGA process). In another embodiment, short wavelength light is used to pattern a photosensitive material such that a subsequent development of the photosensitive material yields the body of the relevant filter element or filter assembly housing (the “LI” of a LIGA process).

A third aspect of the present invention is embodied by an implant having an implant housing and a filter assembly. The implant housing includes a passageway in which the filter assembly is positioned. Components of the filter assembly include at least a first housing and a microfabricated filter element. The first housing includes a first passageway, a flow through the implant passageway is directed through the microfabricated filter element, and the first housing is more rigid than the implant housing at least when the implant is installed in the targeted biological material.

Various refinements exist of the features noted in relation to the third aspect of the present invention. Further features may also be incorporated in the third aspect of the present invention as well. These refinements and additional features may exist individually or in any combination. The microfabricated filter element may be disposed within the first housing or mounted on an open end of the first housing. The filter assembly may include a second housing that is at least partially disposed within the first housing such that the first housing is disposed about the microfabricated filter element, and the position of the microfabricated filter element may be fixed relative to the second housing in any appropriate manner. Further in this regard, the filter element may be disposed within the second housing, or the microfabricated filter element may interface with an open end of the second housing. This second housing may be maintained in a fixed position relative to the first housing in any appropriate manner as well (e.g., press fit, shrink fit, bonded). Moreover, the second housing may also be more rigid than the implant housing at least when the implant is installed in the targeted biological material.

The filter assembly used by the third aspect may further include second and third housings. The second and third housings may be disposed in end-to-end relation, with the microfabricated filter element being disposed between and in contact with each of the second and third housings. Each of the second and third housings may be at least partially disposed within the first housing such that the first housing is disposed about the microfabricated filter element. Preferably, the first, second, and third housings collectively protect and/or support the microfabricated filter element. In this regard, the second and third housings also may be more rigid than the implant housing, at least when the implant is installed in the targeted biological material. In addition, the microfabricated filter element may have first and second primary surfaces that are separated by the spacing limitations noted in relation to the first aspect and between which a plurality of pores extend.

The above-noted second and third housings each may be maintained in a fixed position relative to the first housing in any appropriate manner (e.g., press fit, shrink fit, bonded). The microfabricated filter element may be maintained in a fixed position relative to both the second and third housings in any appropriate manner as well. For instance, the microfabricated filter element may be maintained in a fixed position merely by interfacing with both the second and third housings when positioned within the first housing. Another option would be to mount the microfabricated filter element to the first end of one or both of the second and third housings before disposing the same within the first housing. Preferably, the microfabricated filter element is maintained in a fixed position relative to each of the first, second, and third housings.

### BRIEF DESCRIPTION OF THE SEVERAL VIEWS OF THE DRAWING

FIG. 1 is an exploded, perspective view of one embodiment of a filter assembly having a filter element that is fabricated using at least part of a LIGA process.

FIG. 2 is a perspective view of the filter assembly of FIG. 1 in an assembled condition.

FIG. 3A is an exploded, perspective of another embodiment of a filter assembly having a filter element that is fabricated using at least part of a LIGA process.

FIG. 3B is a perspective view of the filter assembly of FIG. 3A in an assembled condition.

FIG. 4A is an exploded, perspective of another embodiment of a filter assembly having a filter element that is fabricated by using at least part of a LIGA process.

FIG. 4B is a perspective view of the filter assembly of FIG. 4A in an assembled condition.

FIG. 5A is a top view of the filter element used by the filter assemblies of FIGS. 1-4B.

FIG. 5B is a cross-sectional view of the filter element of FIG. 5A, taken along line B-B.

FIG. 6 is one embodiment of a protocol for fabricating the filter element of FIGS. 5A-B using at least part of a LIGA process.

FIG. 7A is one embodiment of a protocol for fabricating a filter assembly housing using at least part of a LIGA process, and that may be used by the filter assemblies of FIGS. 1-4B.

FIG. 7B is another embodiment of a protocol for fabricating a filter assembly housing using at least part of a LIGA process, and that may be used by the filter assemblies of FIGS. 1-4B.

FIG. 8 is a schematic (side view) of one embodiment of an implant filter assembly that may be in the form of any of the filter assemblies of FIGS. 1-4B.

FIG. 9 is a cross-sectional view of one embodiment of an implant, with the implant filter assembly of FIG. 8 being disposed in its hollow interior.

### DETAILED DESCRIPTION OF THE INVENTION

The present invention will now be described in relation to the accompanying drawings that at least assist in illustrating its various pertinent features. Generally, the various devices described herein, or at least one or more components thereof, are microfabricated. There are a number of microfabrication technologies that are commonly characterized as “micromachining,” including without limitation LIGA (Lithographie, Galvonoformung, Abformung), SLIGA (sacrificial LIGA), bulk micromachining, surface micromachining, micro electrodischarge machining (EDM), laser micromachining, 3-D stereolithography, and other techniques. Hereafter, the term “MEMS device,” “microfabricated device” or the like means any such device that is fabricated using a technology that allows realization of a feature size of 10 microns or less. “Microfabrication” thereby means a fabrication technique that allows realization of a feature size of 10 microns or less.

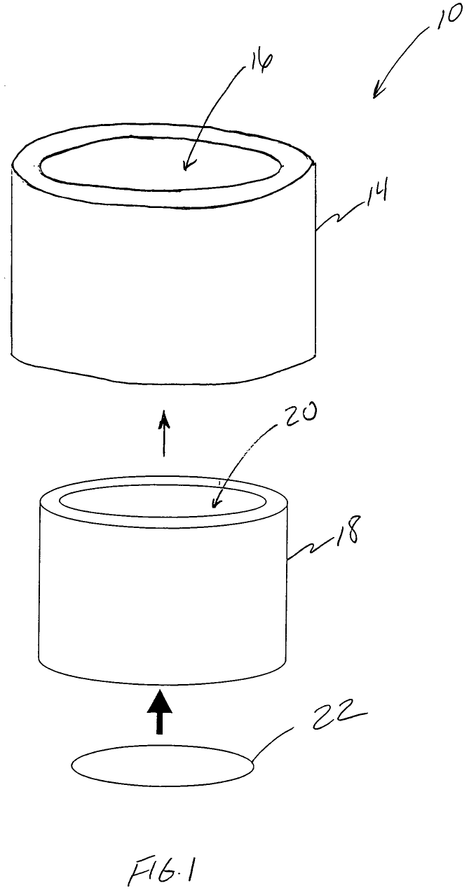

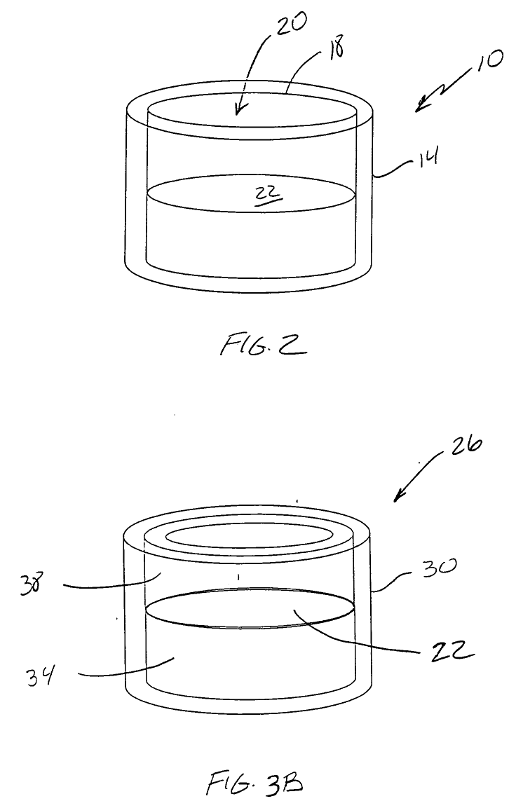

FIGS. 1-2 schematically represent one embodiment of a filter assembly 10 that may be used for any appropriate application (e.g., the filter assembly 10 may be disposed in a flow of any type, may be used to filter a fluid of any type, may be located between any pair of fluid or pressure sources (including where one is the environment), or any combination thereof). Components of the filter assembly 10 include an outer housing 14, an inner housing 18, and a LIGA filter element 22. The position of the LIGA filter element 22 and the inner housing 18 are at least generally depicted within the outer housing 14 in FIG. 2 to show the relative positioning of these components in the assembled condition—not to convey that the outer housing 14 needs to be in the form of a transparent structure. All details of the LIGA filter element 22 and the inner housing 18 are not necessarily illustrated in FIG. 2.

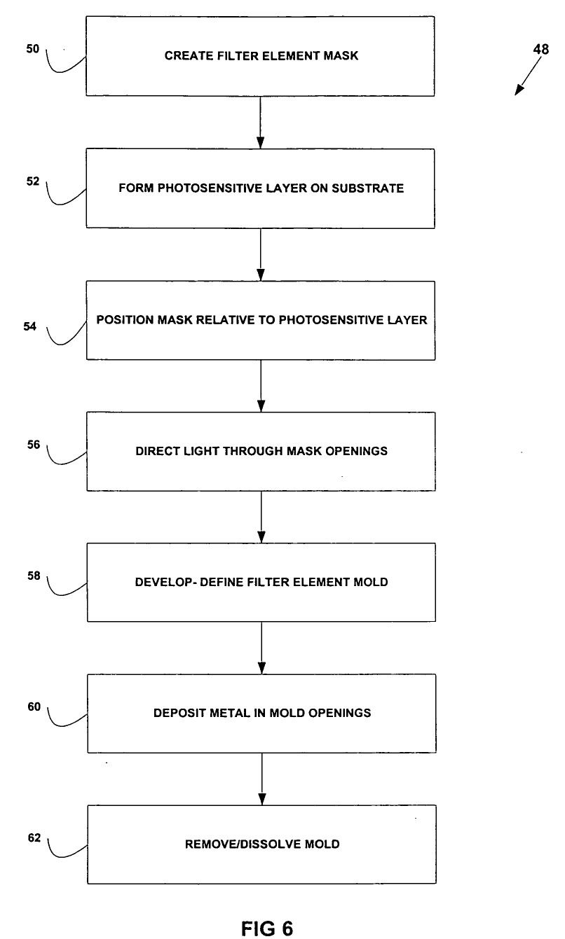

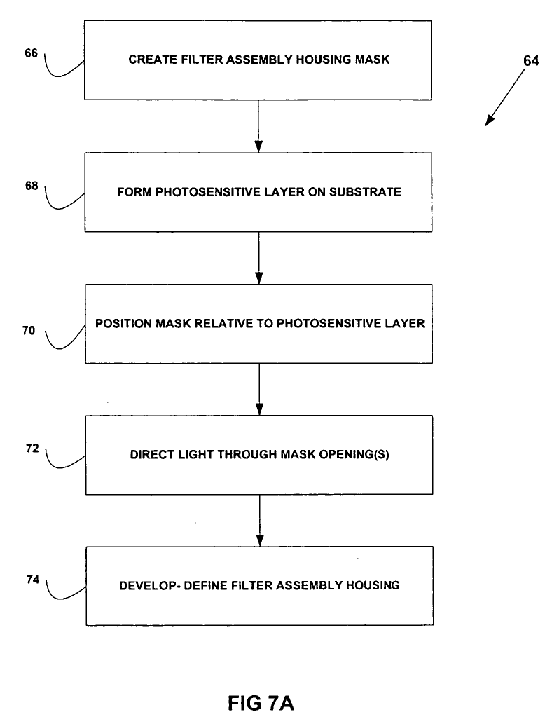

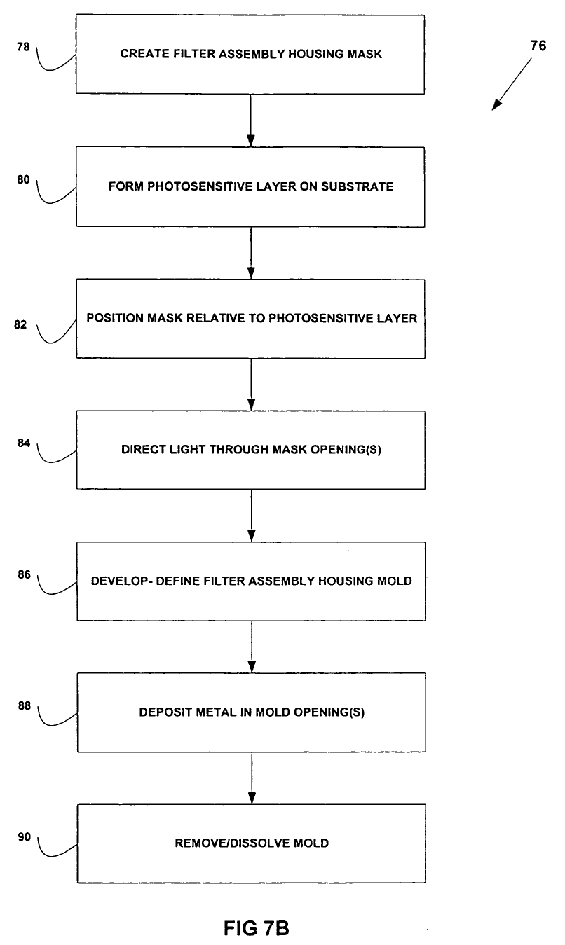

The LIGA filter element 22 is only schematically represented in FIGS. 1-2, provides at least a filtering function, and is microfabricated by using at least part of a LIGA process (e.g., using the “LI” part of a LIGA process and in accordance with the protocol 76 of FIG. 7B that will be discussed in more detail below; using the “LIG” part of a LIGA process and in accordance with the protocol 48 of FIG. 6 that will be discussed in more detail below). Therefore, using “LIGA” in the definition of the filter element 22 means that at least part of a LIGA process is used in the fabrication thereof. The LIGA filter element 22 may be of any appropriate design, size, shape, and configuration, and further may be formed from any material or combination of materials that are appropriate for fabrication by at least part of a LIGA process. The LIGA filter element 22 is at least generally disk-shaped, and has a thickness of no more than about 50 microns in one embodiment, a thickness of no more than about 15 microns in another embodiment, and a thickness of about 5 microns in yet another embodiment.

The primary function of the outer housing 14 and inner housing 18 is to provide structural integrity for the LIGA filter element 22 or to support the LIGA filter element 22, and further to protect the LIGA filter element 22. In this regard, the outer housing 14 and inner housing 18 each will typically be in the form of a structure that is sufficiently rigid to protect the LIGA filter element 22 from being damaged by the forces that reasonably could be expected to be exerted on the filter assembly 10 during its assembly, as well as during use of the filter assembly 10 in the application for which it was designed.

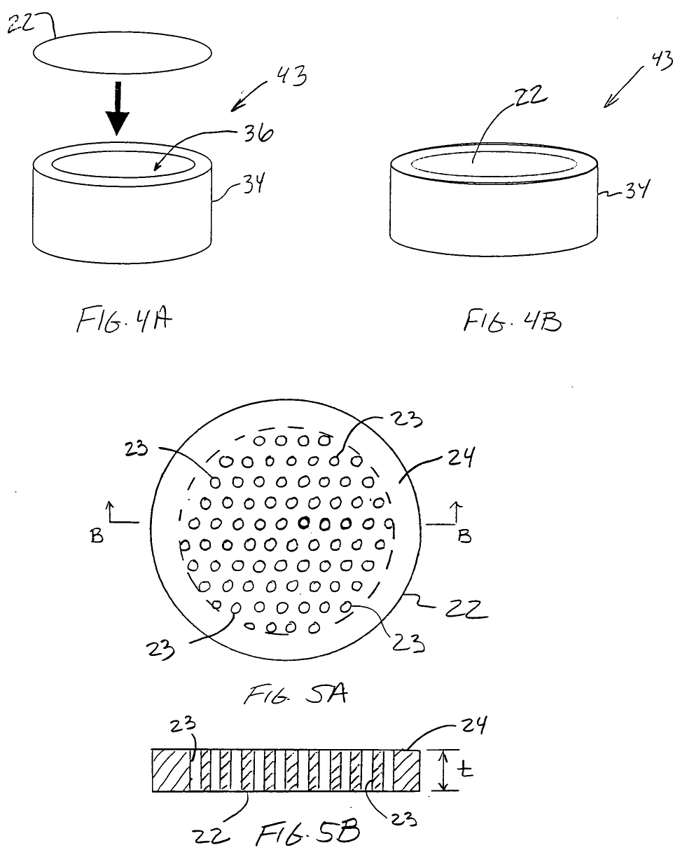

The inner housing 18 includes a hollow interior or a flow path 20 that extends through the inner housing 18 (between its opposite ends in the illustrated embodiment). The LIGA filter element 22 may be disposed within the flow path 20 through the inner housing 18 in any appropriate manner and at any appropriate location within the inner housing 18 (e.g., at any location so that the inner housing 18 is disposed about the LIGA filter element 22). Preferably, the LIGA filter element 22 is maintained in a fixed position relative to the inner housing 18 in any appropriate manner. For instance, the LIGA filter element 22 may be attached or bonded to an inner sidewall of the inner housing 18 or a flange formed on this inner sidewall, a press-fit could be provided between the inner housing 18 and the LIGA filter element 22, or a combination thereof. The LIGA filter element 22 also could be attached to an end of the inner housing 18 in the manner of the embodiment of FIGS. 4A-B that will be discussed in more detail below.

The inner housing 18 is at least partially disposed within the outer housing 14 (thereby encompassing having the outer housing 14 being disposed about the inner housing 18 along the entire length of the inner housing 18, or only along a portion of the length of the inner housing 18). In this regard, the outer housing 14 includes a hollow interior 16 for receiving the inner housing 18, and possibly to provide other appropriate functionality (e.g., a flow path fluidly connected with the flow path 20 through the inner housing 18). The outer and inner sidewalls of the outer housing 14 are preferably cylindrical for flow purposes, as are the outer and inner sidewalls of the inner housing 18, although other configurations may be appropriate. The inner housing 18 may be retained relative to the outer housing 14 in any appropriate manner. For instance, the inner housing 18 may be attached or bonded to an inner sidewall of the outer housing 14, a press-fit could be provided between the inner housing 18 and the outer housing 14, a shrink fit could be provided between the outer housing 14 and the inner housing 18, or any combination thereof.

The inner housing 18 is only schematically represented in FIGS. 1-2, and it may be of any appropriate shape/configuration, of any appropriate size, and formed from any material or combination of materials (e.g., polymethylmethacrylate (PMMA), titanium, and other implantable metals and plastics). Typically its outer contour will be adapted to match the inner contour of the outer housing 14 in which it is at least partially disposed. In one embodiment, the illustrated cylindrical configuration for the inner housing 18 is achieved by cutting an appropriate length from hypodermic needle stock. The inner housing 18 also may be microfabricated into the desired/required shape (e.g., using at least part of a LIGA process). However, any way of making the inner housing 18 may be utilized. It should also be appreciated that the inner housing 18 may include one or more coatings as desired/required as well (e.g., an electroplated metal).

The outer housing 14 is only schematically represented in FIGS. 1-2, and it may be of any appropriate shape/configuration, of any appropriate size, and formed from any material or combination of materials (e.g., polymethylmethacrylate (PMMA), titanium, and other implantable metals and plastics). Typically its outer contour will be adapted to match the inner contour of the housing in which it is at least partially disposed or otherwise mounted. The outer housing 14 also may be microfabricated into the desired/required shape (e.g., using at least part of a LIGA process). However, any way of making the outer housing 14 may be utilized. It should also be appreciated that the outer housing 14 may include one or more coatings as desired/required as well (e.g., an electroplated metal).

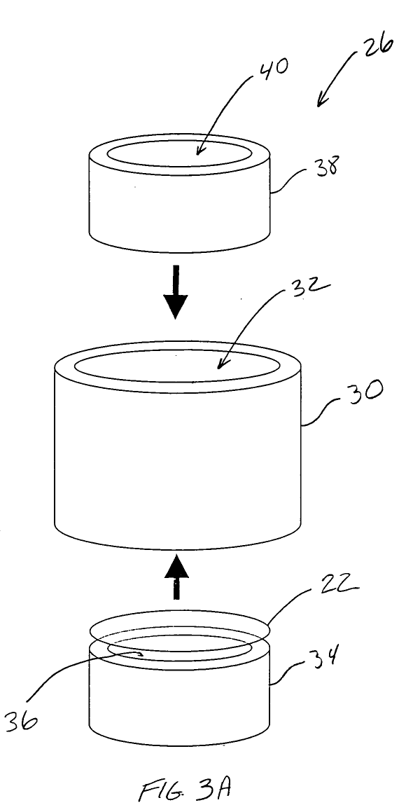

Another embodiment of a filter assembly is illustrated in FIGS. 3A-B (only schematic representations), and is identified by reference numeral 26. The filter assembly 26 may be used for any appropriate application (e.g., the filter assembly 26 may be disposed in a flow of any type, may be used to filter a fluid of any type, may be located between any pair of fluid or pressure sources (including where one is the environment), or any combination thereof). Components of the filter assembly 26 include an outer housing 30, a first inner housing 34, a second inner housing 38, and the above-noted LIGA filter element 22. The LIGA filter element 22 and the inner housings 34, 38 are at least generally depicted within the outer housing 30 in FIG. 3B to show the relative positioning of these components in the assembled condition—not to convey that the outer housing 30 needs to be in the form of a transparent structure. All details of the LIGA filter element 22 and the inner housings 34, 38 are not necessarily illustrated in FIG. 3B.

The primary function of the outer housing 30, first inner housing 34, and second inner housing 38 is to provide structural integrity for the LIGA filter element 22 or to support the LIGA filter element 22, and further to protect the LIGA filter element 22. In this regard, the outer housing 30, first inner housing 34, and second inner housing 38 each will typically be in the form of a structure that is sufficiently rigid to protect the LIGA filter element 22 from being damaged by the forces that reasonably could be expected to be exerted on the filter assembly 26 during its assembly, as well as during use of the filter assembly 26 in the application for which it was designed.

The first inner housing 34 includes a hollow interior or a flow path 36 that extends through the first inner housing 34. Similarly, the second inner housing 38 includes a hollow interior or a flow path 40 that extends through the second inner housing 38. The first inner housing 34 and the second inner housing 40 are disposed in end-to-end relation, with the LIGA filter element 22 being disposed between adjacent ends of the first inner housing 34 and the second inner housing 38. As such, a flow progressing through the first flow path 36 to the second flow path 40, or vice versa, passes through the LIGA filter element 22.

Preferably, the LIGA filter element 22 is maintained in a fixed position relative to each inner housing 34, 38, and its perimeter does not protrude beyond the adjacent sidewalls of the inner housings 34, 38 in the assembled and joined condition. For instance, the LIGA filter element 22 may be bonded to at least one of, and thereby including both of, the first inner housing 34 (more specifically one end thereof) and the second inner housing 38 (more specifically one end thereof) to provide structural integrity for the LIGA filter element 22 (e.g., using cyanoacrylic esters, UV-curable epoxies, or other epoxies). Another option would be to fix the position the LIGA filter element 22 in the filter assembly 26 at least primarily by fixing the position of each of the inner housings 34, 38 relative to the outer housing 30 (i.e., the LIGA filter element 22 need not necessarily be bonded to either of the housings 34, 38). In one embodiment, an elastomeric material may be disposed between the LIGA filter element 22 and the first inner housing 34 to allow the first inner housing 34 with the LIGA filter element 22 disposed thereon to be pushed into the outer cylinder 30 (e.g., the elastomeric material is sufficiently “tacky” to at least temporarily retain the LIGA filter element 22 in position relative to the first inner housing 34 while being installed in the outer housing 30). The second inner housing 38 also may be pushed into the outer housing 30 (before, but more likely after, the first inner housing 34 is disposed in the outer housing 30) to “sandwich” the LIGA filter element 22 between the inner housings 34, 38 at a location that is within the outer housing 30 (i.e., such that the outer housing 30 is disposed about the LIGA filter element 22). The LIGA filter element 22 would typically be contacted by both the first inner housing 34 and the second inner housing 38 when disposed within the outer housing 30. Fixing the position of each of the first inner housing 34 and the second inner housing 38 relative to the outer housing 30 will thereby in effect fix the position of the LIGA filter element 22 relative to the outer housing 30.

Both the first inner housing 34 and second inner housing 38 are at least partially disposed within the outer housing 30 (thereby encompassing the outer housing 30 being disposed about either or both housings 34, 38 along the entire length thereof, or only along a portion of the length of thereof), again with the LIGA filter element 22 being located between the adjacent ends of the first inner housing 34 and the second inner housing 38 and within the outer housing 30. In this regard, the outer housing 30 includes a hollow interior 32 for receiving at least part of the first inner housing 34, at least part of the second inner housing 38, and the LIGA filter element 22 disposed therebetween, and possibly to provide other appropriate functionality (e.g., a flow path fluidly connected with the flow paths 36, 40 through the first and second inner housings 34, 38, respectively). The outer and inner sidewalls of the outer housing 30 are preferably cylindrical for flow purposes, as are the outer and inner sidewalls of the inner housings 34, 38, although other configurations may be appropriate. Both the first inner housing 34 and the second inner housing 38 may be maintained in a fixed position relative to the outer housing 30 in any appropriate manner, including in the manner discussed above in relation to the inner housing 18 and the outer housing 14 of the embodiment of FIGS. 1-2.

Each inner housing 34, 38 is only schematically represented in FIGS. 3A-B, and each may be of any appropriate shape/configuration, of any appropriate size, and formed from any material or combination of materials in the same manner as the inner housing 18 of the embodiment of FIGS. 1-2. Typically the outer contour of both housings 34, 38 will be adapted to match the inner contour of the outer housing 30 in which they are at least partially disposed. In one embodiment, the illustrated cylindrical configuration for the inner housings 34, 38 is achieved by cutting an appropriate length from hypodermic needle stock. The inner housings 34, 38 each also may be microfabricated into the desired/required shape (e.g., using at least part of a LIGA process). However, any way of making the inner housings 34, 38 may be utilized. It should also be appreciated that the inner housings 34, 38 may include one or more coatings as desired/required as well (e.g., an electroplated metal).

The outer housing 30 is only represented in FIGS. 3A-B, and it may be of any appropriate shape/configuration, of any appropriate size, and formed from any material or combination of materials in the same manner as the outer housing 14 of the embodiment of FIGS. 1-2. Typically the outer contour of the outer housing 30 will be adapted to match the inner contour of the housing in which it is at least partially disposed or otherwise mounted. The outer housing 30 may be microfabricated into the desired/required shape (e.g., using at least part of a LIGA process). However, any way of making the outer housing 30 may be utilized. It should also be appreciated that the outer housing 30 may include one or more coatings as desired/required as well (e.g., an electroplated metal).

Another embodiment of a filter assembly is illustrated in FIGS. 4A-B (only schematic representations), and is identified by reference numeral 43. The filter assembly 43 may be used for any appropriate application (e.g., the filter assembly 43 may be disposed in a flow of any type, may be used to filter a fluid of any type, may be located between any pair of fluid or pressure sources (including where one is the environment), or any combination thereof). Components of the filter assembly 43 include the above-noted housing 34 and the LIGA filter element 22 from the embodiment of FIGS. 3A-B. In the case of the filter assembly 43, the LIGA filter element 22 is attached or bonded to one end of the housing 34 (e.g., using cyanoacrylic esters, UV-curable epoxies, or other epoxies). The filter assembly 43 may be disposed within an outer housing in the manner of the embodiments of FIGS. 1-3B, or could be used “as is.”

The LIGA filter element 22 used by the filter assemblies 10, 26, and 43 is illustrated in more detail in FIGS. 5A-B. A plurality of pores 23 extend through the entire thickness of the LIGA filter element 22 (represented by a dimension “t” in FIG. 5B) throughout a desired/required region. In the illustrated embodiment, a perimeter region 24 of the LIGA filter element 22 is devoid of any pores 23. This may facilitate a desired interface with a housing of a filter assembly that is using the LIGA filter element 22 (e.g., inner housing 18 in the case of filter assembly 10 (e.g., for interfacing with a flange, lip or other support(s) on the inner sidewall of the inner housing 18); for interfacing with the ends of the inner housings 34, 38 in the case of the filter assembly 26; for interfacing with the end of the housing 34 in the case of the filter assembly 43). However, the pores 23 could be disposed throughout the entirety of the LIGA filter element 22.

The pores 23 may be of any appropriate size and shape. Cylindrical pores 23 are preferred for at least some applications for flow purposes. Any number of pores 23 may be utilized and to define any desired/required open area or porosity for the filtering region of the LIGA filter element 22 (that region having pores 23, which is disposed inwardly of the perimeter region 24 in the illustrated embodiment). In one embodiment, the open area or porosity of the LIGA filter element 22 is at least about 50%, and is defined as the ratio of the collective area of the pores 23 in the filtering region to the area of the filtering region at either of the two primary surfaces or faces of the LIGA filter element 22. Any distribution of pores 23 may be utilized as well (e.g., equally spaced or otherwise). It should be appreciated that at least some of the pores 23 on the perimeter of the filtering region 24 could possibly be in the form of partial pores 23.

At least part of a LIGA process may be used in the fabrication of the LIGA filter element 22. LIGA processes in general are addressed in a book by M. J. Madou, entitled “Fundamentals of Microfabrication,” (2^{nd} ed. 2002) (CRC Press, Boca Raton, Fla.), the entire disclosure of which is incorporated by reference in its entirety herein. Any known aspect of LIGA fabrication may be employed in the fabrication of the LIGA filter element 22 and/or any corresponding filter assembly housing described herein, as appropriate. One benefit of the short wavelength light used by a LIGA fabrication technique is the high degree of dimensional control associated with LIGA. Specifically, not only can the size of the individual pores 23 be controlled within a tight tolerance using short wavelength light (e.g., within about 20 nanometers), but the position of these individual pores on the LIGA filter element 22 can be controlled within this same tight tolerance. The potential for two or more pores 23 being disposed in overlapping relation (to collectively define a larger pore) using LIGA to fabricate the LIGA filter element 22 can thereby be greatly reduced and possibly totally eliminated. This may be important for a particular application, such that the potential for a particle of greater than a certain size being able to pass through the LIGA filter element 22 is greatly reduced.

One embodiment of a protocol for fabricating the filter element 22 using at least part of a LIGA process (specifically, the “LIG” portion of a LIGA process) is illustrated in FIG. 6 and is identified by reference numeral 48. A filter element mask of any appropriate material (e.g., gold) is created by step 50 to define the desired configuration for the LIGA filter element 22, including without limitation the size, shape, and positioning of the various pores 23. For instance, an opening may exist in the filter element mask that corresponds with the space between the various pores 23 for a positive resist system, or openings may exist in the filter element mask that coincide with the various pores 23 for a negative resist system. In any case, a photosensitive layer of any appropriate material is formed in any appropriate manner on any appropriate substrate (e.g., silicon) in step 52 (e.g., spinning, casting, bonding one or more sheet onto the substrate in a laminated fashion). Representative materials for this photosensitive layer include without limitation polymethylmethacrylate (PMMA).

The filter element mask is then appropriately positioned relative to the photosensitive layer pursuant to step 54 of the protocol 48. Typically, the filter element mask will be positioned close to the photosensitive layer. In any case, light is then directed through the opening(s) in the filter element mask pursuant to step 56. Typically extremely short wavelength light will be used, such as x-rays (e.g., 2-10 Angstroms), extreme UV rays (e.g., 10-14 nanometers), or deep UV rays (e.g., 150-300 nanometers). Extreme UV rays (wavelengths on the order of about 10-14 nm) and x-rays (wavelengths on the order of 10 Angstroms or less) are preferred for at least some applications based upon the increase in dimensional control that is realized as the magnitude of the wavelength is reduced.

The light associated with step 56 of the protocol 48 changes the nature of the photosensitive layer in regions that correspond with the mask opening(s). The photosensitive layer is then developed to define a filter element mold pursuant to step 58. For instance, any portion of the photosensitive layer that was exposed to the light in step 56 may be removed (e.g., dissolved) by execution of step 58 for the case of a positive resist system (any portion of the photosensitive layer that was not exposed to the light in step 56 may be removed by the execution of step 58 for the case of a negative resist system). What is then left is a plurality of structures that will ultimately define the pores 23 in the LIGA filter element 22 (specifically the various flow paths through the LIGA filter element 22). An appropriate material is then appropriately placed into the open space between these structures. Stated another way, this material is deposited in the opening(s) in the filter element mold defined by step 58. Typically step 60 will be in the form of electroplating, where the desired metal is deposited into the space in the filter element mold (e.g., between the above-noted structures of developed photosensitive material). Thereafter, the filter element mold is removed in any appropriate manner, such as by dissolving the developed photosensitive material. This then creates a plurality of open areas corresponding with the various pores 23 of the LIGA filter element 22, and further provides a body for the LIGA filter element 22 from the material deposition of step 60.

FIG. 7A illustrates one embodiment of a protocol that may be used to fabricate a filter assembly housing (e.g., to fabricate any of the housings 14, 18, 30, 34, 38) and that is identified by reference numeral 64. Generally, this protocol 64 uses the “LI” portion of a LIGA process. A filter assembly housing mask of any appropriate material (e.g., gold) is created by step 66 to define the desired configuration for the filter assembly housing. In the case of a cylindrical filter assembly housing and for a positive resist system, the mask may include a circular opening to correspond with its hollow interior, as well as an annular opening that is spaced from the outer perimeter of this circular opening (the distance between these two openings thereby defining the thickness of the sidewall of the cylindrical filter assembly housing). In the case of a cylindrical filter assembly housing and for a negative resist system, the mask may include an annular opening that corresponds with the thickness of the sidewall of the cylindrical filter assembly housing.

A photosensitive layer of any appropriate material is formed in any appropriate manner on any appropriate substrate (e.g., silicon) pursuant to step 68 of the protocol 64 (e.g., spinning, casting, bonding one or more sheet onto the substrate in a laminated fashion). Representative materials for this photosensitive layer include without limitation polymethylmethacrylate (PMMA). The filter assembly housing mask (step 66) is then appropriately positioned relative to the photosensitive layer (step 68) pursuant to step 70. Typically, the filter assembly housing mask will be positioned close to the photosensitive layer. In any case, light is then directed through the opening(s) in the filter assembly housing mask pursuant to step 72. Typically short wavelength light will be used for step 72, such as x-rays, extreme UV rays, or deep UV rays. Extreme UV rays (wavelengths on the order of about 10-14 nm) and x-rays (wavelengths on the order of 10 Angstroms or less) are preferred for at least some applications based upon the increase in dimensional control that is realized as the magnitude of the wavelength is reduced. That is, there may be a need for a filter assembly housing to be fabricated within a tight tolerance (e.g., within about 0.5 μm for the case of where a microfabricated filter element is to be press fit within a filter assembly housing), and that is achievable with short wavelength light in the manner. Using short wavelength light to fabricate a cylindrical filter assembly housing thereby may produce inner and/or outer walls for the filter assembly housing that have a desired degree of verticality, a wall thickness with minimal variance along the entire length of the filter assembly housing, or both.

The light associated with step 72 of the protocol 64 changes the nature of the photosensitive layer in regions that correspond with the mask opening(s). The photosensitive layer is then developed to define the filter assembly housing pursuant to step 74 (e.g., to dissolve the exposed photoresist in the case of a positive resist system; to dissolve the unexposed photoresist in the case of a negative resist system). That is, the material that defines the filter assembly housing in the case of the protocol 64 is the developed photosensitive material. Photosensitive materials that will likely be sufficiently rigid (once developed) for purposes of any of the housings used by the filter assemblies 10, 26, and 48 include without limitation PMMA. The protocol 64 of FIG. 7A could be adapted to fabricate the LIGA filter element 22 (e.g., making appropriate changes to the mask in step 66), where the “body” of the LIGA filter element 22 would then be in the form of a photosensitive material.

FIG. 7B illustrates another embodiment of a protocol that may be used to fabricate a filter assembly housing (e.g., to fabricate any of the housings 14, 18, 30, 34, 38), that is identified by reference numeral 76, and that is at least somewhat similar to the protocol 48 of FIG. 6 on filter element fabrication. Generally, this protocol 76 uses the “LIG” portion of a LIGA process. A filter assembly housing mask of any appropriate material (e.g., gold) is created by step 78 to define the desired configuration for the filter assembly housing. In the case of a cylindrical filter assembly housing and for a negative resist system, the mask may include a circular opening to correspond with its hollow interior, as well as an annular opening that is spaced from the outer perimeter of this circular opening (the distance between these two openings thereby defining the thickness of the sidewall of the cylindrical filter assembly housing). In the case of a cylindrical filter assembly housing and for a positive resist system, the mask may include an annular opening that corresponds with the thickness of the sidewall of the cylindrical filter assembly housing.

A photosensitive layer of any appropriate material is formed in any appropriate manner on any appropriate substrate (e.g., silicon) pursuant to step 80 of the protocol 76 (e.g., spinning, casting, bonding one or more sheet onto the substrate in a laminated fashion). Representative materials for this photosensitive layer include without limitation polymethylmethacrylate (PMMA). The filter assembly housing mask (step 78) is then appropriately positioned relative to the photosensitive layer (step 80) pursuant to step 82. Typically, the filter assembly housing mask will be positioned close to the photosensitive layer. In any case, light is then directed through the opening(s) in the filter assembly housing mask pursuant to step 84. Typically short wavelength light will be used for step 84, such as x-rays, extreme UV rays, or deep UV rays. Extreme UV rays (wavelengths on the order of about 10-14 nm) and x-rays (wavelengths on the order of 10 Angstroms or less) are preferred for at least some applications based upon the increase in dimensional control that is realized as the magnitude of the wavelength is reduced and as previously noted.

The light associated with step 84 of the protocol 76 changes the nature of the photosensitive layer in regions that correspond with the mask opening(s). The photosensitive layer is then developed to define a filter assembly housing mold pursuant to step 86. For instance, that portion of the photosensitive layer that was not exposed to the light in step 84 may be removed (e.g., dissolved) by execution of step 86 for the case of a negative resist system, while the portion of the photosensitive layer that was exposed to the light in step 86 may be removed (e.g., dissolved) for the case of a positive resist system. For the case of a cylindrical filter assembly housing, what is then left is a solid cylindrical plug, and an annular wall that is disposed about and spaced from the perimeter of this cylindrical plug (this space corresponding with the wall thickness of the filter assembly housing that is ultimately defined). Stated another way, what is left is an annular opening that extends down through the photosensitive layer. An appropriate material is then appropriately placed into this annular opening. Stated another way, this material is deposited in the annular opening of the filter assembly housing mold. Typically step 88 will be in the form of electroplating, where the desired metal is deposited into the space in the filter assembly housing mold (e.g., between the above-noted structures of developed photosensitive material). Thereafter, the filter assembly housing mold is removed in any appropriate manner, such as by dissolving the developed photosensitive material. This then creates a hollow interior or flow path through the filter assembly housing, and further provides a body for the filter assembly housing from the material deposition of step 88.

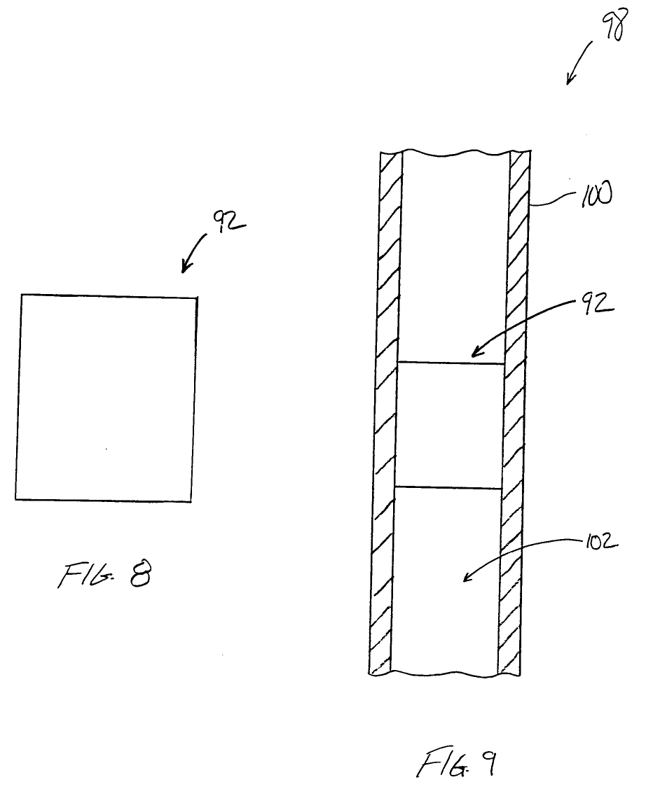

One particularly desirable application for the filter assemblies 10, 26, and 43 is for use in implant. An implant filter assembly is schematically presented in FIG. 8 and is identified by reference numeral 92. The implant filter assembly 92 may be in the form of any of the filter assemblies 10, 26, or 43, but in any case includes at least one LIGA filter element 22 and at least one housing. This housing supports/protects the LIGA filter element 22 when utilized for an implant application. Moreover, this housing is more rigid than the implant housing in which it is disposed, at least when the implant housing is installed in a targeted biological material. That is, the implant housing may be formed of a material whose rigidity changes when it is exposed to a biological material (e.g., it may become less rigid when exposed to the biological material).

FIG. 9 discloses one embodiment of an implant 98. The implant 98 includes an implant housing 100 of a configuration that is appropriate for the particular application (e.g., the illustrated portion of the housing 100 may be of any appropriate configuration, as may its opposite ends (not shown), and that may be formed from any appropriate material or combination of materials. However, the implant housing 100 should be biologically compatible with the targeted application. Specifically, the implant housing 100 should be biologically compatible with the biological material in which it will be disposed. The implant 98 may be used for any relevant application, such as for relieving pressure. For instance, the implant 98 may be used to address an elevated intraocular pressure in the anterior chamber of a patient's eye. The implant 98 may also be appropriate for addressing an elevated pressure within a patient's inner ear. Again, it should be appreciated that the configuration of the implant 98 may be tailored as desired/required for the target application. Representative configurations for the implant 98 include without limitation those disclosed by any of U.S. Pat. No. 3,788,327, entitled “Surgical Implant Device”; U.S. Pat. No. 5,743,868, entitled “Corneal Pressure-Regulating Implant Device”; or U.S. Pat. No. 5,807,302, entitled “Treatment of Glaucoma”, as well as that disclosed by U.S. Patent Application Publication No. US2003/0212383 A1, entitled “System and Methods for Reducing Intraocular Pressure”, the entire disclosures of each of these documents. being incorporated by reference in their entirety herein.

At least during use of the implant 98 (i.e., at least when installed in the targeted biological material), at least one housing of the implant filter assembly 92 is more rigid than the implant housing 100. That is, the implant housing 100 may become less rigid when exposed to a biological material, and at this time the housing(s) of the implant filter assembly 92 is more rigid than the implant housing 100. Each housing of the implant filter assembly 92 is preferably more rigid than the implant housing 100, at least when the implant 98 is installed. In the case of the filter assembly 10 of FIGS. 1-2, at least the outer housing 14 would then be more rigid than the implant housing 100 in the noted manner (although the inner housing 18 is also preferably more rigid than the implant housing 100 in the noted manner). In the case of the filter assembly 26 of FIGS. 3A-B, at least the outer housing 30 would be more rigid than the implant housing 100 in the noted manner (although the inner housings 34, 38 are also preferably more rigid than the implant housing 100 in the noted manner). In the case of the filter assembly 43 of FIGS. 4A-B, the housing 34 would be more rigid than the implant housing 100 in the noted manner.

The implant housing 100 includes a hollow interior or passageway 102 (e.g., for accommodating a flow (continuous and/or intermittent) of a relevant fluid). This passageway 102 is filtered by incorporating the implant filter assembly 92 into this passageway 102. A flow through the passageway 102 of the implant 98 is preferably directed through the implant filter assembly 92 (and thereby through at least one LIGA filter element 22).

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A method for making an implant, comprising the steps of:
fabricating a filter element, wherein said fabricating step comprises forming a plurality of pores using at least part of a LIGA process; and
disposing said filter element in a passageway of an implant housing, and wherein a flow through said passageway is directed through said filter element.

2. A method, as claimed in claim 1, further comprising the step of:
assembling said filter element and a first housing into a filter assembly, wherein said assembling step comprises maintaining said filter element in a fixed position relative to said first housing, wherein said disposing step comprises disposing said filter assembly in said passageway of said implant housing after said assembling step.

3. A method, as claimed in claim 2, wherein:
said first housing is more rigid than said implant housing when said implant is installed in a biological material.

4. A method, as claimed in claim 2, wherein:
said assembling step comprises disposing said filter element within said first housing.

5. A method, as claimed in claim 2, wherein:
said assembling step comprises mounting said filter element on a first open end of said first housing.

6. A method, as claimed in claim 2, wherein:
said filter element comprises first and second primary surfaces that are separated by a distance of no more than about 50 μm, and wherein said pores extend between said first and second primary surfaces.

7. A method, as claimed in claim 2, further comprising the step of:
fabricating said first housing using at least part of a LIGA process.

8. A method, as claimed in claim 2, further comprising the step of:
fabricating said first housing, wherein said fabricating said first housing step comprises exposing a photosensitive material to light selected from the group consisting of x-rays, extreme ultraviolet rays, deep ultraviolet rays, or any combination thereof.

9. A method, as claimed in claim 1, further comprising the step of:
assembling said filter element, a first housing, and a second housing into a filter assembly, wherein said assembling step comprises disposing said filter element within said second housing, wherein said assembling step further comprises disposing at least a portion of said second housing within said first housing, wherein said disposing said filter element step comprises disposing said filter assembly in said passageway of said implant housing.

10. A method, as claimed in claim 9, further comprising the step of:
maintaining said second housing in a fixed position relative to said first housing.

11. A method, as claimed in claim 10, further comprising the step of:
maintaining said filter element in a fixed position relative to said second housing.

12. A method, as claimed in claim 9, further comprising the step of:
maintaining said filter element in a fixed position relative to said second housing.

13. A method, as claimed in claim 9, wherein:
said filter element comprises first and second primary surfaces that are separated by a distance of no more than about 50 μm, and wherein said pores extend between said first and second primary surfaces.

14. A method, as claimed in claim 9, wherein:
said disposing at least a portion of said second housing step is executed after said disposing said filter element within said second housing step.

15. A method, as claimed in claim 9, wherein:
said first and second housings are each more rigid than said implant housing when said implant is installed in a biological material.

16. A method, as claimed in claim 1, further comprising the step of:
assembling said filter element, a first housing, a second housing, and a third housing into a filter assembly, wherein said assembling step comprises disposing a first end of said second housing within said first housing, disposing a first end of said third housing within said first housing, and locating said filter element between said first end of said second housing and said first end of said third housing, and wherein said disposing said filter element step comprises disposing said filter assembly in said passageway of said implant housing.

17. A method, as claimed in claim 16, further comprising the step of:
maintaining each of said second and third housings in a fixed position relative to said first housing.

18. A method, as claimed in claim 17, further comprising the step of:
maintaining said filter element in a fixed position relative to each of said second and third housings.

19. A method, as claimed in claim 16, further comprising the step of:
maintaining said filter element in a fixed position relative to each of said first, second, and third housings.

20. A method, as claimed in claim 16, further comprising the step of:
positioning said filter element on said first end of said second housing before executing said disposing a first end of said second housing step.

21. A method, as claimed in claim 16, wherein:
said filter element comprises first and second primary surfaces that are separated by a distance of no more than about 50 μm, and wherein said pores extend between said first and second primary surfaces.

22. A method, as claimed in claim 16, wherein:
said first, second, and third housings are each more rigid than said implant housing when said implant is installed in a biological material.

23. A method for making an implant, comprising the steps of:
fabricating a filter element comprising exposing a first photosensitive material to light selected from the group consisting of x-rays, extreme ultraviolet rays, deep ultraviolet rays, or any combination thereof;
fabricating a first housing comprising exposing a second photosensitive material to light selected from the group consisting of x-rays, extreme ultraviolet rays, deep ultraviolet rays, or any combination thereof;
assembling said filter element and said first housing into a filter assembly; and
disposing said filter assembly in a passageway of an implant housing, wherein a flow through said passageway passes through said filter element.

24. A method, as claimed in claim 23, wherein:
said first housing is more rigid than said implant housing when said implant is installed in a biological material.

25. A method, as claimed in claim 23, wherein:
said assembling step comprises disposing said filter element within said first housing.

26. A method, as claimed in claim 23, wherein:
said assembling step comprises mounting said filter element on a first open end of said first housing.

27. A method, as claimed in claim 23, wherein:
said first housing comprises a first passageway, wherein said filter element is disposed within said first passageway, and wherein said first housing is disposed about said filter element.

28. A method, as claimed in claim 23, wherein:
said filter element comprises first and second primary surfaces that are separated by a distance of no more than about 50 μm, and wherein a plurality of pores extend between said first and second surfaces.

29. A method, as claimed in claim 23, wherein:
said filter assembly further comprises a second housing.

30. A method, as claimed in claim 29, wherein said assembling step further comprises:
fixing a position of said filter element relative to said second housing;
disposing at least a portion of said second housing within said first housing such that said first housing is disposed about said filter element; and
fixing a position of said second housing relative to said first housing.

31. A method, as claimed in claim 30, wherein:
said fixing a position of said filter element step is executed before said disposing at least a portion of said second housing step.

32. A method, as claimed in claim 30, wherein:
said first and second housings are each more rigid than said implant housing when said implant is installed in a biological material.

33. A method, as claimed in claim 30, wherein:
said filter element comprises first and second primary surfaces that are separated by a distance of no more than about 50 μm, and wherein a plurality of pores extend between said first and second surfaces.

34. A method, as claimed in claim 29, further comprising the step of:
fabricating said second housing comprising exposing a third photosensitive material to light selected from the group consisting of x-rays, extreme ultraviolet rays, deep ultraviolet rays, or any combination thereof.

35. A method, as claimed in claim 29, wherein said assembling step further comprises the steps of:
disposing said filter element within said second housing; and
disposing said second housing at least partially within said first housing.

36. A method, as claimed in claim 23, wherein said assembling step further comprises the steps of:
disposing said filter element within said first housing;
disposing a second housing of said filter assembly at least partially within said first housing; and
disposing a third housing of said filter assembly at least partially within said first housing, wherein said filter element is disposed between said second and third housings.

37. A method, as claimed in claim 36, wherein said assembling step further comprises the step of:
positioning said filter element on an end of said second housing before executing said disposing a second housing step, wherein said first housing is disposed about said filter element after said disposing a second housing step.

38. A method, as claimed in claim 36, wherein:
said second and third housings are each more rigid than said implant housing when said implant is installed in a biological material.

39. A method, as claimed in claim 36, wherein:
said filter element comprises first and second primary surfaces that are separated by a distance of no more than about 50 μm, and wherein said pores extend between said first and second surfaces.

40. An implant, comprising:
an implant housing comprising a passageway; and
a filter assembly disposed in said passageway, wherein said filter assembly comprises a microfabricated filter element and a first housing, wherein said first housing comprises a first passageway, wherein a flow through said passageway is directed through said microfabricated filter element, and wherein said first housing is more rigid than said implant housing when said implant is installed in a biological material.

41. An implant, as claimed in claim 40, wherein:
said microfabricated filter element is disposed within said first housing.

42. An implant, as claimed in claim 40, wherein:
said microfabricated filter element is mounted on a first open end of said first housing.

43. An implant, as claimed in claim 40, wherein:
said filter assembly further comprises a second housing that is at least partially disposed within said first housing, wherein a position of said microfabricated filter element is fixed relative to said second housing, and wherein said first housing is disposed about said microfabricated filter element.

44. An implant, as claimed in claim 43, wherein:
said microfabricated filter element is disposed within said second housing.

45. An implant, as claimed in claim 43, wherein:
said microfabricated filter element interfaces with a first open end of said second housing, wherein said first open end is disposed within said first housing.

46. An implant, as claimed in claim 43, wherein:
a position of said second housing is fixed relative to said first housing.

47. An implant, as claimed in claim 40, wherein:
said filter assembly further comprises a second housing and a third housing, wherein said second and third housings are disposed in end-to-end relation with said microfabricated filter element being disposed therebetween and in contact with each of said second and third housings, and wherein said second and third housings are each at least partially disposed within said first housing such that said first housing is disposed about said microfabricated filter element.

48. An implant, as claimed in claim 47, wherein:
a position of each of said second and third housings is fixed relative to said first housing.
